(19) United States
(12) Patent Application Publication
(10) Pub. No.: US 2004/0068246 A1
(43) Pub. Date: Apr. 8, 2004

(54) Disposable garments with improved elastic filament placement control and methods of producing same

(75) Inventors: Brian M. Rose, Neenah, WI (US); Sandra A. Richlen, Black Creek, WI (US)

Correspondence Address: Schwegman, Lundberg, Woessner & Kluth, P.A., P.O. Box 2938, Minneapolis, MN, 55402, US

(73) Assignee: Kimberly-Clark Worldwide, Inc.

(21) Appl. No.: 10/263,120

(22) Filed: Oct. 2, 2002

(51) Int. Cl. A61F 13/15; A61F 13/20

(52) U.S. Cl. 604/385.27

(57) ABSTRACT

The present invention provide a disposable garment comprising a laminated panel having at least two layers secured together with panel adhesive, the laminated panel further containing elastic filaments to provide active elastic control, the elastic filaments having end Portions secured in place with a securing element located a predetermined distance from an edge of the laminated panel, wherein the active elastic control extends up to the securing element. In one embodiment, the securing element is an adhesive bead. The present invention further provides a method for forming the disposable garment.

### FIELD

The present invention relates to elastic filament placement control, and, in particular, to improved elastic filament placement control.

### BACKGROUND

Disposable garments with elasticized leg openings are known in the art. See, for example, U.S. Pat. No. 4,437,860 to Sigl et al (1984). However, control of elastic filament placement in areas that are oscillated in and out of machine direction (MD) is difficult. Specifically, current processes involve cutting such elastic filaments within the product, and allowing them to snap back. However, there is a risk in damaging the product laminate with this method. Furthermore, in the process of snapping back, these filaments can retract too far, i.e., creep back. As a result, a gap in the leg opening or crotch area develops that does not have active elastic control. Such a gap limits the ability to control fit in the final product, thus reducing leakage protection.

Therefore, there is a need in the art to provide a product and method of making the product that improves elastic filament placement as well as product fit and leakage protection.

### SUMMARY

The present invention provides, in one embodiment, a disposable garment comprising a laminated panel having at least two layers secured together with panel adhesive, the laminated panel further containing elastic filaments to provide active elastic control, the elastic filaments having end portions secured in place with a securing element located a predetermined distance from an edge of the laminated panel, wherein the active elastic control extends up to the securing element. In one embodiment, the securing element is an adhesive bead.

In one embodiment, the disposable garment has active elastic control to within at least five (5) cm (2 in) of the edge of the garment. In other embodiments, there is active elastic control to within less than about five (5) cm down to about 0.3 cm (0.13 in). In a particular embodiment, the elastic filaments are located in a curved area of the garment, such as adjacent to an opening.

In another embodiment, the present invention provides a method for forming a disposable garment comprising, simultaneously, applying an adhesive bead to an inner layer as it is being unwound, unwinding an outer layer and oscillating elastic filaments between the inner layer and outer layer; placing the inner layer on top of the outer layer, the outer layer having elastic filaments contained therein, to produce a laminate; and cutting the elastic filaments located outside the laminate with a cutting means, wherein the elastic filaments snap back to an outer edge of the adhesive bead.

The resulting product provides increased control of product fit by keeping elastic tension in the desired position. The product further provides a corresponding increase in product leakage protection due to the presence of the securing element that prevents or minimizes elastic retraction. Furthermore, since the elastic filaments are cut outside the laminate, product performance issues otherwise caused by marking, cutting or damaging the laminate are eliminated. This is important to consumer perception of potential damage caused by the cutting operation.

The present invention is useful for any garment requiring active elastic control, including garments requiring elastic constructions for various openings, such as arms, legs, neck, waist or head. As a result, the invention can be incorporated into any type of disposable garment requiring elastic or stretchable filaments, including, but not limited to absorbent articles such as diapers, training pants, adult incontinence and feminine care garments, or any other garment that requires elastic tension and placement in specific areas.

### BRIEF DESCRIPTION OF THE DRAWINGS

FIG. 1 is a simplified plan view showing three sections of an exemplary disposable garment after being cut from a web of material, together with back panel elastic filaments secured by an adhesive bead in accordance with one embodiment of the present invention.

FIG. 2 is a more detailed plan view of the garment of FIG. 1, with portions broken away for clarity, in accordance with one embodiment of the present invention.

FIG. 3 is a plan view of a web of disposable garment material from which individual panels for garments are subsequently cut showing conventional placement of adhesive and elastic filaments.

FIG. 4 is a plan view of a web of disposable garment material from which individual panels for garments are subsequently cut showing placement of adhesive and elastic filaments in accordance with one embodiment of the present invention.

FIG. 5 is a schematic illustration of an exemplary process for forming a disposable garment in accordance with one embodiment of the present invention.

FIG. 6 is a block diagram of a process for forming a disposable garment in accordance with one embodiment of the present invention.

FIG. 7 is a simplified illustration of a disposable garment in accordance with one embodiment of the present invention.

### DETAILED DESCRIPTION

In the following detailed description of the preferred aspects, reference is made to the accompanying drawings, which form a part hereof, and in which is shown by way of illustration specific preferred aspects in which the invention may be practiced. These aspects are described in sufficient detail to enable those skilled in the art to practice the invention, and it is to be understood that other aspects may be utilized and that chemical, mechanical, procedural and other changes may be made without departing from the spirit and scope of the present invention. The following detailed description is, therefore, not to be taken in a limiting sense, and the scope of the present invention is defined only by the appended claims, along with the full scope of equivalents to which such claims are entitled.

The present invention provides a disposable garment comprising a laminated web portion containing one or more oscillated elastic filaments having end portions extending beyond the laminated web portion. The present invention also provides a method for making a disposable garment that includes cutting the elastic filaments outside of the laminated web portion.

Various definitions used throughout the specification are provided first, followed by a description of various embodiments of the present invention.

### Definitions

As used herein, the term “disposable absorbent garment” refers to a garment that typically includes a bodyside liner and an absorbent element. The absorbent element typically includes an absorbent material. Often, such garments include a body chassis for supporting the absorbent element, which itself can include multiple components, such as an absorbent core, intake/distribution layer and so forth. Such garments include, for example, incontinence undergarments, which are typically configured with a self-supporting waist band, or diapers, and the like, which can be secured on the user with tabs, belts and the like. The body chassis can include a liquid permeable top sheet or film secured to an outer cover or backsheet, i.e., liner, which can be liquid permeable or impermeable, depending on whether an additional backsheet, i.e., barrier, is provided. Typically, the absorbent element is disposed between the body chassis and the user. The body chassis can take many forms, including for example, a pant-like or underwear type undergarment described herein, which includes a self-supporting waistband extending circumferentially around the waist of the user. Alternatively, the body chassis can be a diaper or like garment, which is secured around the user with various fastening means or devices known by those of skill in the are, including for example and without limitation tabs, belts and the like. Preferably, the chassis includes elastic regions formed along the edges of the crotch region and around the leg openings, so as to form a gasket with the user's crotch and legs.

As used herein, the term “nonwoven web” means a structure or a web of material that has been formed without use of traditional fabric forming processes, such as weaving or knitting, to produce a structure of individual fibers or threads that are intermeshed, but not in an identifiable, repeating manner. Non-woven webs can be formed by a variety of conventional processes such as, for example, meltblowing processes, spunbonding processes, film aperturing processes and staple fiber carding processes.

As used herein, the term “machine direction (MD)” refers to the direction of travel of the forming surface onto which fibers are deposited during formation of a nonwoven fibrous web or product elements are deposited during assembly of product.

As used herein, the term “cross-machine direction (CD)” refers to a direction that is essentially perpendicular to MD, i.e., “side-to-side.”

As used herein, the term “active elastic” refers to elastic filaments or strands that are gathered during use, i.e., under residual tension, thus providing active elastic control in desired areas of a disposable garment.

As used herein, the term “nonactive elastic” (or “inactive” or “dead” elastic) refers to elastic filaments or strands that do not retract during use, thus providing no elastic control in the area of the disposable garment where they are present. In most embodiments of the present invention, dead elastic filaments are located primarily or exclusively outside the garment.

As used herein, the term “snapback” or “creep back” or “retraction” refers to the action of elastic filaments after being cut in conventional disposable garments, wherein the elastic filaments retract back into the garment a sufficient distance from the edge of the adhesive, so as to become inactive elastic.

It should be understood that the term “longitudinal,” as used herein, means of or relating to length or the lengthwise direction, and in particular, the direction running between the front and back of the user. The term “laterally,” as used herein means situated on, directed toward or running from side to side, and in particular, a direction running from the left to the right of a user. The terms “inner,” and “outer” as used herein are intended to indicate the direction relative to the user wearing an absorbent garment over the crotch region. For example, the term “inner” refers to a “bodyside,” i.e., side closest to the body of the user, while the term “outer” refers to a “garment side.”

### Description of the Embodiments

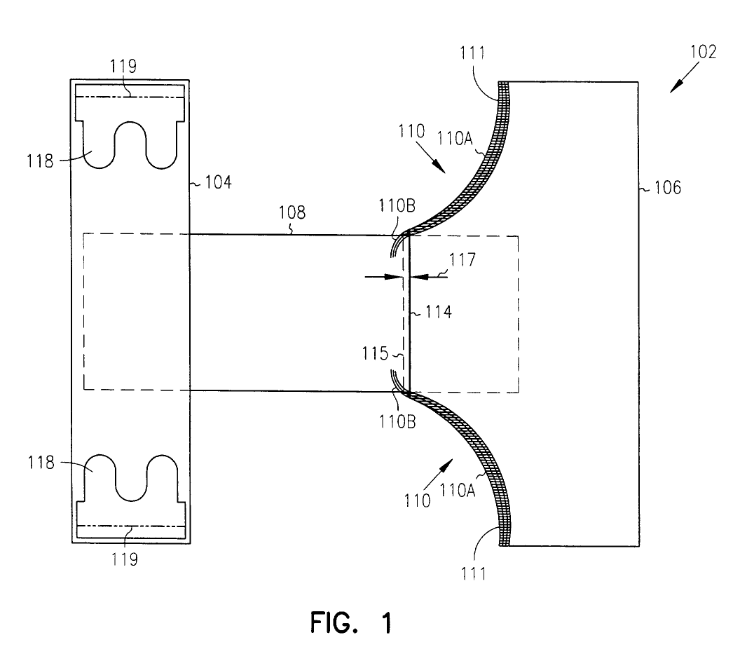

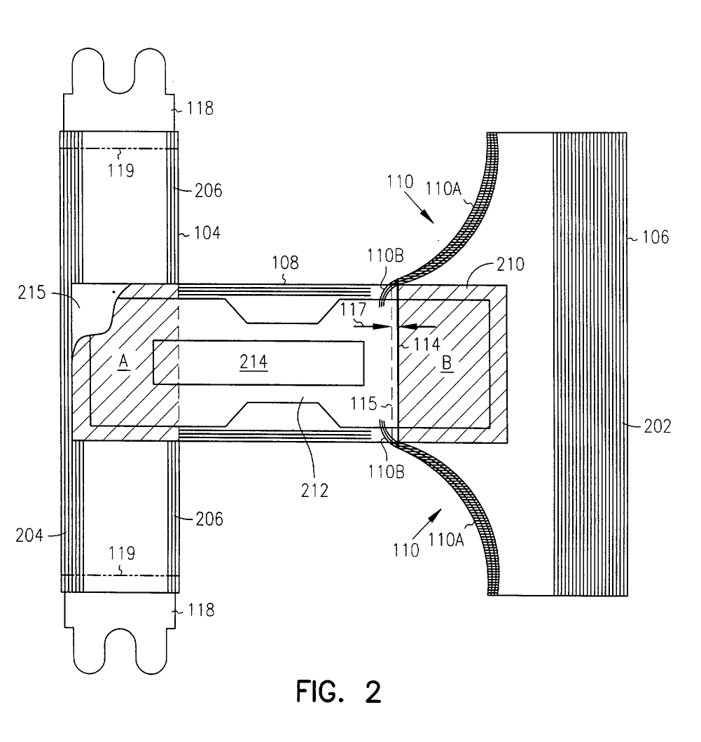

FIG. 1 provides a simplified plan view of one embodiment of the present invention, wherein the disposable garment is an adult incontinence garment 102 that includes a bodyfit chassis (hereinafter “chassis”). In this embodiment, the chassis includes form-fitting body coverage elements, including a laminated front panel 104, a laminated back panel 106, with a crotch region or insert 108 extending between the panels. In this embodiment, the laminated back panel 106 has two sets of back panel elastic filaments or strands 110, with each set having three elastic filaments apiece, although the invention is not so limited. Any number of filaments can be used in each set and any number of sets of back panel elastic filaments 110 can be used. In this embodiment, each set is adjacent to a curved leg edge 111, although the invention is not so limited. Essentially, the back panel elastic filaments 110 can be located in any desired position within the garment. Additionally, in most embodiments, other sets of elastic filaments are also present, as shown in FIG. 2, which are designed to maintain a comfortable snug fit against the body of the user.

Each set of back panel elastic filaments 110 includes an active elastic portion 110A contained within the laminated back panel 106 and an inactive elastic portion (i.e., an “end portion” comprising the dead filaments extending outside of the laminated back panel 106) 110B. Each active elastic portion 110A extends to a securing element 114 that, in this embodiment, runs substantially parallel to an inner edge 115 of the laminated back panel 106 at a predetermined distance 117. In other embodiments, the securing element 114 is positioned to capture elastic filaments at any desired location within the product. The inactive elastic portion 110B can be removed later in a separate process, if desired.

Any number of back panel elastic filaments 110 can be used. In one embodiment, one elastic filament is used. In most embodiments, more than one elastic filament is used, such as 3-6 elastic filaments, to provide the desired tension.

The laminated back panel 106 is designed to be worn in the back and the laminated front panel 104 is designed to be worn in the front of the user. In this embodiment, the laminated front panel 104 includes a set of refastenable members 118 that are bonded to the outer portion of the laminated front panel 104 as shown, although the invention is not so limited. The refastenable members 118 can be secured to the product in locations other than the laminated front panel via any means known in the art. Such refastenable members 118 can include, but are not limited to, pressure sensitive tapes, hook and loop-type securing means, any type of bonding means, and so forth. In another embodiment, the refastenable members are replaced with non-refastenable members that are designed to be secured in place only once. In yet other embodiments, there are no refastenable members 118 and the side seams are bonded or stitched together to form a pull-on article.

Perforation lines 119 are located in the laminated front panel 104 and used to separate the garment into a “front portion” and “rear portion” prior to use. As a result, once separated, the rear portion includes not only the entire laminated back panel 106, but also a section of the laminated front panel 104 up to the perforation line 119, such that the refastenable members 118 become part of the rear portion during use.

Additional detail of the adult incontinence garment 102 of FIG. 1 is shown in FIG. 2. As can be seen, the laminated rear panel 106 further comprises a set of rear panel waist elastic filaments 202 (hereinafter “rear waist filaments”). In the embodiment shown, there are 20 rear waist filaments, although any suitable number can be used, depending on the design and configuration of the garment. In one embodiment, two (2) to 35 rear waist filaments are used. In another embodiment, 15 to 25 rear waist filaments are used. In an exemplary embodiment, 19 rear waist filaments are used.

Additionally, the laminated front panel 104 contains a set of front panel waist elastic filaments 204 (hereinafter “front waist filaments”). The laminated front panel 104 additionally contains front panel leg elastic filaments 206 (hereinafter “front leg filaments”) that extend underneath the insert 108. Again, although this embodiment shows six (6) front waist filaments 204 and three (3) front leg filaments 206 in each set, any suitable number of filaments can be used in each area as desired for the particular design. In one embodiment, up to about 15 front waist filaments 204 are used. In one embodiment, up to about eight front leg filaments 206 are used.

The individual layers (or inner and outer layers) of the laminated front panel 104 and laminated back panel 106 can be made of any suitable material, such as a woven, nonwoven, plastic, or the like. In one embodiment, the individual layers are a 0.3 to 0.7 osy spunbond material. In a particular embodiment, the individual layers are a 0.6 osy spunbond material.

In the embodiment shown in FIG. 2, the insert 108 is comprised of a fluid impervious backing sheet or outer cover 210, a fluid pervious facing sheet or liner 215, an absorbent core 212 and an intake/distribution layer 214. The outer cover 210 serves as a fluid barrier and can be made from any suitable liquid impermeable material or a material treated to be liquid impermeable. In one embodiment, the outer cover 210 is a laminate comprised of an inner liner layer and an outer film layer, such as a polyethylene film. In one embodiment, “breathable stretch thermal laminate” (BSTL) is used for the outer cover 210. In an alternative embodiment the outer cover is an opaque sheet of material with an embossed or matte surface that is about one mil thick, although the invention is not so limited. In another alternative embodiment, the outer surface is made of extensible materials, such as necked, pleated (or micropleated) or creped nonwovens, including spunbond polypropylenes, bonded carded webs, or laminates of nonwovens and films that are necked, pleated or creped so as to allow the outer cover to extend with minimal force. For example, a suitable extensible material is a 60% necked, polypropylene spunbound having a basis weight of about 1.2 osy. For a further description of extensible materials, see U.S. patent application Ser. No. 09,855,182, filed on May 14, 2001, entitled, “Absorbent Garment with Expandable Absorbent Element,” commonly assigned, and hereby incorporated herein by reference. The outer cover can also be made of nonwovens, films, or composites of films and nonwovens.

The liner 215 may be any soft, flexible porous sheet that permits the passage of fluids therethrough, including, but not limited to, hydrophobic or hydrophilic nonwoven webs, wet strength papers, spunwoven filament sheets, and so forth. In one embodiment, the inner bodyside surface is made from spunwoven polypropylene filaments with spot embossing, further including a perforated surface or suitable surfactant treatment to aid fluid transfer.

The absorbent core or absorbent batt 212 located between the outer cover and liner serves to absorb liquids, as is known in the art and can be made from any suitable material. The absorbent batt can be can be any material that tends to swell or expand as it absorbs exudates, including various liquids and/or fluids excreted or exuded by the user. For example, the absorbent material can be made of airformed, airlaid and/or wetlaid composites of fibers and high absorbency materials, referred to as superabsorbents. Superabsorbents typically are made of polyacrylic acids, such as DOW 2035 available from DOW Chemical of Midland, Mich. The fibers can be fluff pulp materials, such as Alliance CR-1654, or any combination of crosslinked pulps, hardwood, softwood, and synthetic fibers. Airlaid and wetlaid structures typically include binding agents, which are used to stabilize the structure. Other absorbent materials, alone or in combination, and including webs of carded or air-laid textile fibers, multiple plys of creped cellulose wadding, various super absorbent materials, various foams, such as synthetic foam sheets, absorbent films, and the like may also be used. The batt may also be slightly compressed or embossed in selected areas as desired. Various acceptable absorbent materials are disclosed in U.S. Pat. No. 5,147,343, entitled, “Absorbent Products Containing Hydrogels With Ability To Swell Against Pressure,” U.S. Pat. No. 5,601,542, entitled “Absorbent Composite,” and U.S. Pat. No. 5,651,862, entitled, “Wet Formed Absorbent Composite,” all of which are commonly assigned and hereby incorporated herein by reference. Furthermore, the properties of high-absorbency particles can range from about zero (0) to about 100%, and the proportion of fibrous material from about zero (0) to about 100%.

In one embodiment, the absorbent batt is a folded absorbent material made of fibrous absorbent materials with relatively high internal integrity, including for example one made with thermoplastic binder fibers in airlaid absorbents, e.g., pulp, bicomponent binding fibers, and superabsorbents, which have higher densities in the folded regions. The higher density and resulting smaller capillary size in these regions promotes better wicking of the liquid. Better wicking, in turn, promotes higher utilization of the absorbent material and tends to result in more uniform swelling throughout the absorbent material as it absorbs the liquid. The intake/distribution layer 214 is made from any suitable material to increase the weight of fluid intake retention.

The individual layers of the laminated front and back panels, 104 and 106, respectively, are combined into laminated layers using any suitable type of adhesive means known in the art that can maintain a bond during use. Such adhesives are generally tacky upon application to the first sheet and remain so for an amount of time sufficient to allow for placement of the desired components onto the first sheet, such as the elastic filaments and the second sheet. In one embodiment, an adhesive designated as “H2717” manufactured by Bostik-Findley, Inc., having offices in Milwaukee, Wis., is used.

The individual layers of the insert 108 are also combined into laminated layers using a similar type of adhesive. With regard to the application of adhesive for the insert 108, in the embodiment shown in FIG. 2, adhesive is applied to sections A and B of the insert 108.

Other details of conventional construction and materials of disposable garments are understood in the art and will not be discussed in detail herein. See, for example, U.S. Pat. No. 4,437,860 to Sigl, commonly assigned, which is hereby incorporated herein by reference.

The securing element 114 that holds the inactive elastic portion 110B in place can be placed in any desired location within the garment as long as it can perform its intended function. In this way, active elastic control can be provided in any desired location. In the embodiment shown in FIG. 2, the securing element 114 is substantially parallel to and located a predetermined distance 117 from an outer edge 115 of the laminated back panel 106.

In one embodiment, the predetermined distance 117 is about five (cm) (two (2) in), such that the disposable garment has active elastic control to within at least about five (5) cm of the edge of the garment. In another embodiment, the predetermined distance is less than about five (5) cm. In yet another embodiment, the predetermined distance is less than about 2.5 cm. In yet another embodiment, the predetermined distance is less than about 1.2 cm. In yet another embodiment, the predetermined distance is less than about 0.6 cm. In yet another embodiment, the predetermined distance is approximately 0.32 cm (0.125 in).

The securing element 114 can be any suitable type of elastic securing means designed to prevent elastic filaments from snapping back. Such elastic securing means include, but are not limited to, an adhesive bead, an ultrasonic weld, heat weld, any type of tape (e.g., pressure sensitive tape), stitching, and the like.

In embodiments utilizing an adhesive bead, any suitable type of adhesive can be used and applied in any manner to produce a suitable width. In one embodiment, the adhesive bead is the same type of adhesive used to laminate the various layers, but applied in a manner to produce a width of between about 0.2 cm (0.63 in) and 0.6 cm (0.25 in), although the invention is not so limited. In a particular embodiment, the adhesive bead is about 0.3 cm (0.1 in) wide. The actual width is not of particular importance, as long as the back panel elastic filaments 110 are held in place at the desired location, i.e., as the entire width of the securing element 114 is essentially the dividing line between the active elastic portion 110A and the inactive elastic portion 110B. The adhesive bead can be applied continuously or intermittently, as desired.

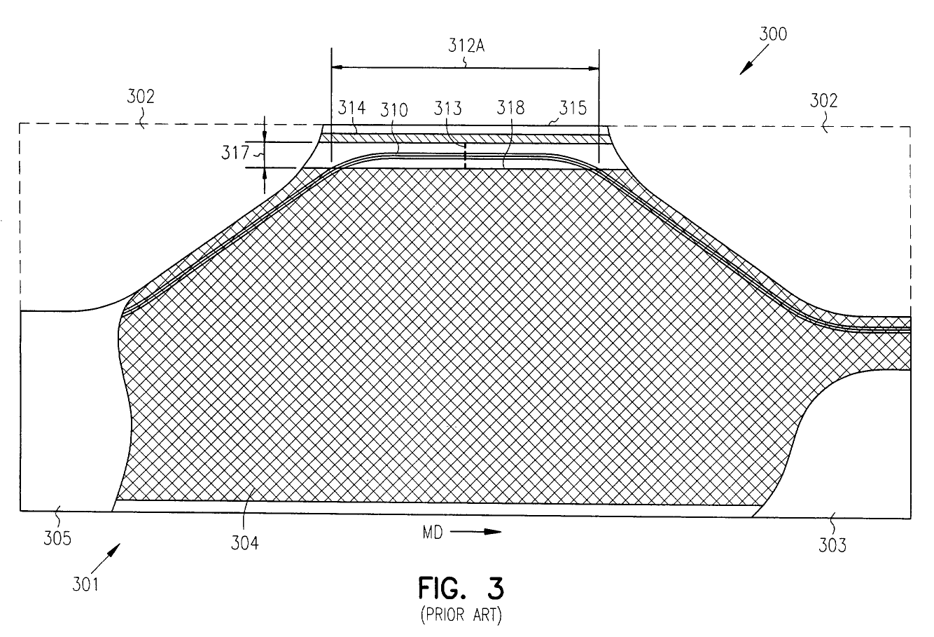

FIG. 3 is a plan view of a web of disposable garment material from which individual garments are subsequently cut, showing conventional placement of elastic filaments in which the elastic filaments are not pulled outside of the laminate. As FIG. 3 demonstrates, a web of back panel outer layer material 300 is shaped by removal of leg cutout sections 302, through use of a leg cutout die, leaving a back panel section 301. As shown in the cutaway portions, the back panel section 301 is comprised of an outer layer 303 on which panel adhesive 304 has been applied, as well as an inner layer 305 that is laminated on top of the outer layer 303 and panel adhesive 304. An edge seal 314 has been applied substantially parallel to an inner edge 315 of the outer layer 303. After the panel adhesive 304 and edge seal 314 have been applied, but prior to being laminated, the back panel elastic filaments 310 are oscillated in and out of MD on the panel adhesive 304, such that an outer section 312A of the elastic filaments 310, i.e., section that is substantially in MD, remains completely contained within the outer layer 303, although is located outside the area of the panel adhesive 304. In other words, the filaments that comprise the outer section 312A lie in a “channel” 317 that is bound on one side by the edge seal 314 and by the outer edge 318 of the panel adhesive 304 on the other side. The inner layer 305 is then laid on top of the outer layer 303 to create the laminated back panel 301 that again has the filaments 310 located completely inside the two laminated layers. The filaments 310 are then cut by cutting along a cut line 313 into the laminate starting at approximately the edge seal 314, through the laminate, through the elastic filaments 310 and again through the laminate to approximately the inner edge 318 of the panel adhesive 304. As the panel adhesive 304 is not sufficient to hold the ends of the elastic filaments 310 in place, the elastic filaments creep back into the laminated back panel an uncontrollable distance. As a result, portions of the leg opening in the final product do not have active control, thus reducing product fit and minimizing leakage control. Additionally, the resulting product has a damaged appearance to the consumer, since the garment itself has been cut.

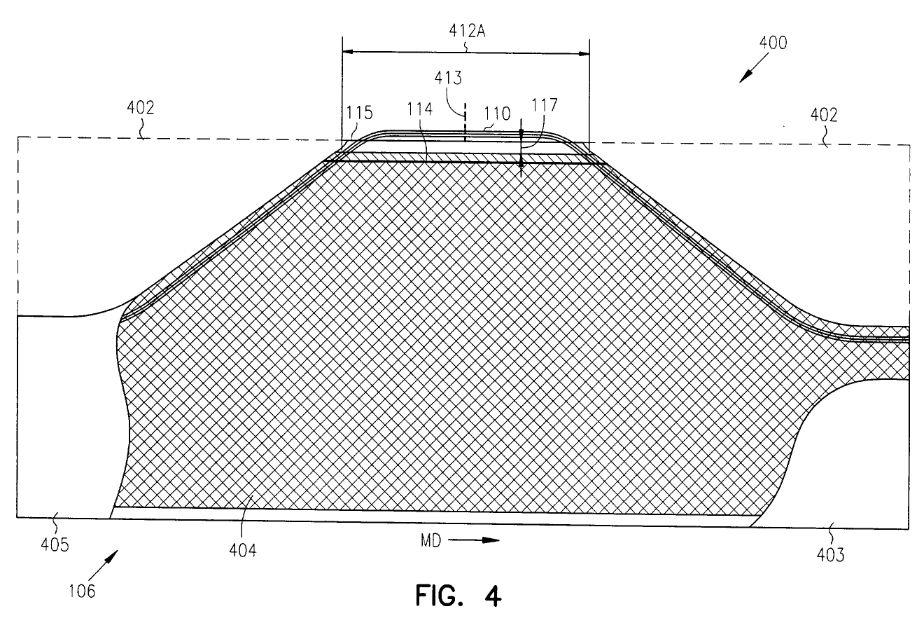

FIG. 4 is a plan view of a web of disposable garment material from which individual garments are subsequently cut, showing placement of the securing element 114 and back panel elastic filaments 110 discussed herein. As FIG. 4 demonstrates, a web of back panel outer layer material 400 is shaped by removal of leg cutout sections 402 through use of a leg cutout die, leaving only the laminated back panel 106. As shown in the cutaway portions, the laminated back panel 106 is comprised of an outer layer 403 on which panel adhesive 404 has been applied, as well as an inner layer 405 that is laminated on top of the outer layer 403. Additionally, in this embodiment, the securing element 114 has been applied substantially parallel to the inner edge 115 of the outer layer 403 at a predetermined distance 117. In this particular embodiment, the securing element 114 is also contiguous with the panel adhesive 404, or, more precisely, an inside edge of the panel adhesive 404, although the invention is not so limited. After the panel adhesive 404 and securing element 114 have been applied, but prior to being laminated, the back panel elastic filaments 110 are oscillated in and out of MD on the inner surface of the outer layer 403 in a substantially sinusoidal pattern, such that an outer section 412A of the back panel elastic filaments 110, i.e., section that is substantially in MD, remains outside the outer layer 403. The inner layer 405 is then laid on top of the outer layer 403 to create the laminated back panel 106 containing the partially laminated filaments 110. The filaments 110 are cut along a cut line 413 through the filaments 110 only, causing them to snap back only to the securing element 114 such that the placement of the filaments 110 is controlled. As a result, the leg opening fits securely on the user, thus providing maximum leakage protection. Additionally, no cut is made to the laminated back panel 106 as with the conventional garment (FIG. 3).

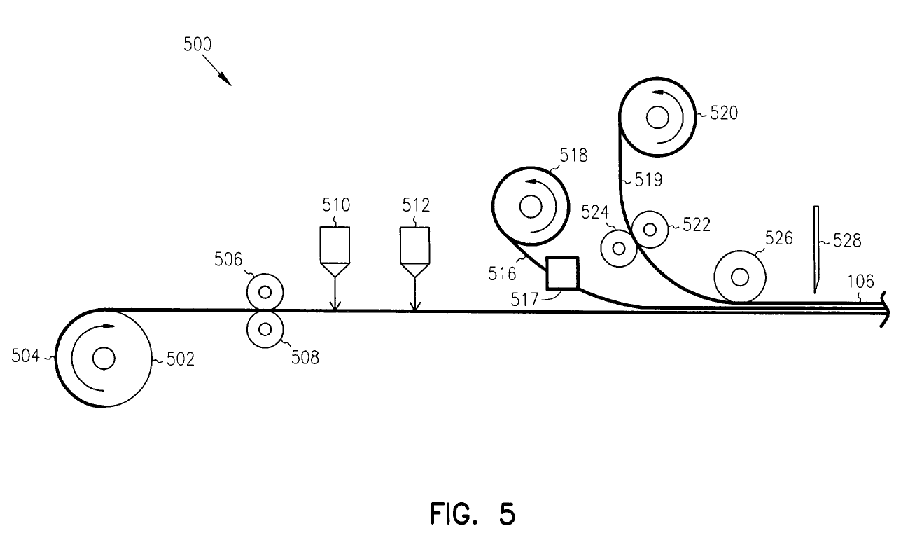

FIG. 5 is a schematic illustration of an exemplary process for forming a disposable garment in one embodiment of the present invention. The process 500 begins when a supply roll 502 having interfacing material, i.e., inner layer 504, is unwound and passed through feed rolls 506 and 508. Panel or lamination adhesive is applied from an adhesive applicator 510, such as a meltblown spray adhesive applicator. Note that the inner layer can be any suitable width and the adhesive pattern width can also vary depending on the product form and grade.

In this embodiment, the securing element is an adhesive bead. The adhesive bead is applied from an adhesive bead applicator 512 at any suitable rate. In one embodiment, the adhesive bead is applied at a rate of about five (5) to 100 gsm using elastic attachment adhesive. In a specific embodiment, the adhesive bead is applied at a rate of about 30 gsm. In one embodiment, the bead is positioned to within about 0.3 cm (0.13 in) of the edge of the web and contiguous with the edge of the back panel lamination adhesive as described herein. Leg elastics 516 are unwound from a feed roll 518 and then applied with a leg elastic oscillator 517 at a suitable rate and degree of elongation. In one embodiment, the leg elastics range from about 470 to 1540 decitex and are applied at an elongation of about 150 to 300%. At this point the outer layer 519 is unwound from an outer layer supply roll 520 and passed through feed rolls 522 and 524. The entire back panel is laminated together, including the portion of the elastics filaments contained within the laminate at a main tacker roll 526. Directly after the main tacker roll 526 a cutting means 528 cuts the portion of the leg elastics extending beyond the laminate to separate them, thus producing a laminated back panel 106. In one embodiment, a flex knife or pinch flex cutter is used. In another embodiment ultrasonics are used. In yet another embodiment a heat laser is used. After being cut, the elastics snap back to the adhesive bead edge and do not creep back into the product past this point.

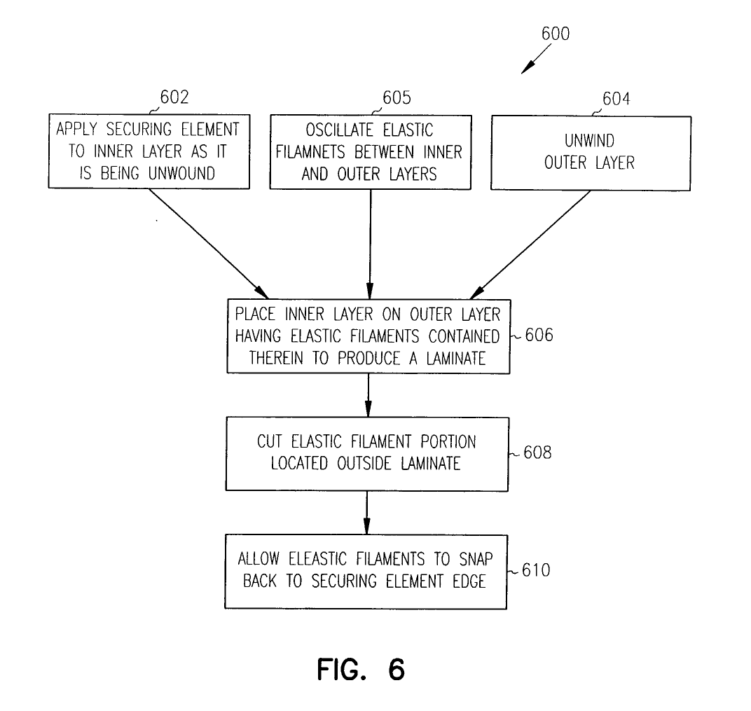

FIG. 6 is a block diagram of a process for forming a disposable garment in one embodiment of the present invention. The process begins by applying 602 a securing element to an inner layer as it is being unwound. Simultaneously, an outer layer is unwound 604 while elastic filaments are oscillated 605 between the inner and outer layers. The inner layer is then placed 606 on top of the outer layer having elastic filaments contained therein to produce a laminate. The elastic filament portion located outside the laminate is then cut 608. As a result, the elastics snap back 610 to the securing element edge. In one embodiment, the securing element is an adhesive bead.

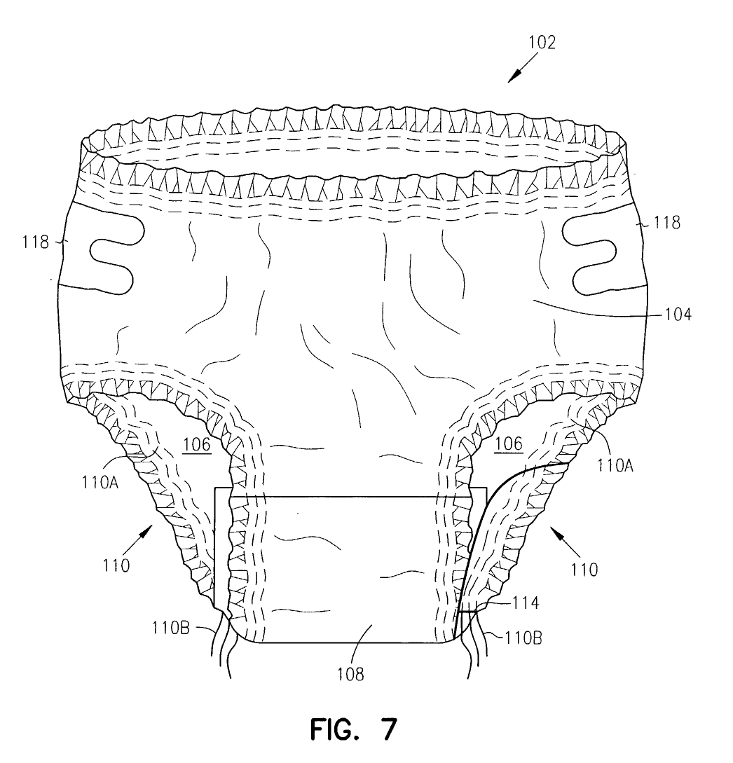

FIG. 7 provides a simplified illustration of an exemplary adult incontinence garment 102 having a back portion comprising the laminated back panel 106 and a small section of the laminated front panel 104 that houses the refastenable members 118. The inactive elastic portions 110B are enlarged for clarity, and again, can be removed in a subsequent cutting process, if desired. The insert 108 and the back panel elastic filaments 110 comprising the active elastic portion 110A and the inactive elastic portion 110B are also shown. As can be seen most clearly in the cut-away portion of FIG. 7, active elastic control is provided essentially throughout the entire leg opening by the active elastic portion 110A of the elastic filaments 110, which is held in place by the securing element 114.

Although the invention has been described primarily in terms of absorbent articles such as diapers, training pants and adult incontinence garments, the present invention is useful for any type of garment requiring elastic constructions for various openings, such as arms, legs, neck, waist or head. Specifically, the invention can be incorporated into any type of disposable garment requiring elastic or stretchable filaments, including, but not limited to, any other garment that requires elastic tension and placement in specific areas, such as hospital garments (e.g., surgical gowns, caps or shoe covers), disposable pajamas, disposable patient gowns, shower caps, hairnets, laboratory coats, and so forth. The present invention is also useful for garments requiring elastic tension at locations other than openings, such as the crotch area, e.g., absorbent articles such as feminine napkins.

Use of the securing element as described herein near the edge of the product serves to control elastic placement and control snapback. Unlike conventional products, the elastics in this invention are oscillated both between and outside the spunbond layers, allowing the cutting process to take place completely outside the resulting laminate.

The present invention provides significant advantages over other disposable garments and methods for manufacture thereof. Unlike prior art products, since the product itself is not cut at the time the elastics are cut, consumers do not perceive that there is damage to the product. Furthermore, since there is essentially continuous active elastic around the leg openings, a good fit and gasketing of the garment are maintained, which thereby improves leakage protection of the garment and the resulting dignity and comfort of the user.

All publications, patents, and patent documents cited in the specification are incorporated by reference herein, as though individually incorporated by reference. In the case of any inconsistencies, the present disclosure, including any definitions therein, will prevail.

Although specific aspects have been illustrated and described herein, it will be appreciated by those of ordinary skill in the art that any arrangement that is calculated to achieve the same purpose may be substituted for the specific aspect shown. This application is intended to cover any adaptations or variations of the present invention. Therefore, it is manifestly intended that this invention be limited only by the claims and the equivalents thereof.

What is claimed is:

1. A disposable garment comprising a laminated panel having at least two layers secured together with panel adhesive, the laminated panel further containing elastic filaments to provide active elastic control, the elastic filaments having end portions secured in place with a securing element located a predetermined distance from an edge of the laminated panel, wherein the active elastic control extends up to the securing element.

2. The disposable garment of claim 1 wherein the securing element is an adhesive bead.

3. The disposable garment of claim 2 wherein the adhesive bead is substantially parallel to the edge of the laminated panel and contiguous with the panel adhesive.

4. The disposable garment of claim 2 wherein the adhesive bead comprises a series of longitudinally spaced apart concentrations of adhesive.

5. The disposable garment of claim 2 wherein the adhesive bead is a continuous bead.

6. The disposable garment of claim 2 wherein the adhesive bead is about 0.2 to 0.6 cm wide.

7. The disposable garment of claim 2 wherein the adhesive bead is applied at a rate of about five (5) to 100 gsm.

8. The disposable garment of claim 1 wherein the securing element is selected from the group consisting of an ultrasonic weld, heat weld, tape, stitching and any combination thereof.

9. The disposable garment of claim 3 wherein the predetermined distance is within at least about 5 cm from the edge of the laminated panel.

10. The disposable garment of claim 3 wherein the predetermined distance is less than 5 cm from the edge of the laminated panel.

11. The disposable garment of claim 3 wherein the predetermined distance is less than about 2.5 cm from the edge of the laminated panel.

12. The disposable garment of claim 3 wherein the predetermined distance is within at least about 1.2 cm from the edge of the laminated panel.

13. The disposable garment of claim 3 wherein the predetermined distance is within approximately 0.3 cm from the edge of the laminated panel.

14. The disposable garment of claim 1 wherein the laminated panel is a back panel having leg openings and the elastic filaments are located adjacent to the leg openings.

15. The disposable garment of claim 1 wherein the laminated panel is made from a nonwoven web.

16. The disposable garment of claim 1 wherein the disposable garment is an adult incontinence garment, diaper or training pants.

17. The disposable garment of claim 1 wherein the disposable garment is a feminine napkin.

18. The disposable garment of claim 1 wherein there are 3 to 6 elastic filaments.

19. The disposable garment of claim 1 wherein the elastic filaments are used in a curved area.

20. A product comprising two substrates laminated together, wherein one or more elastic filaments are partially laminated between the two substrates, further wherein an adhesive bead controls placement of the one or more elastic filaments within the two substrates.

21. The product of claim 20 wherein end portions of the elastic filaments are located outside the two substrates.

22. The product of claim 21 wherein the two substrates are an inner layer and an outer layer and the elastic filaments are secured between the inner layer and the outer layer.

23. The product of claim 20 wherein the two substrates are part of a disposable garment.

24. The product of claim 23 wherein the one or more elastic filaments are located adjacent to an arm opening, leg opening, neck opening, waist opening or head opening.

25. The product of claim 23 wherein the disposable garment is an absorbent article.

26. The product of claim 25 wherein the absorbent article is selected from the group consisting of a diaper, training pants, adult incontinence garment and feminine napkin.

27. A laminated material having at least two layers, wherein the at least two layers are secured together with adhesive, the laminated material further having elastic filament end portions secured in place with an adhesive bead to prevent snap back.

28. The laminated material of claim 27 wherein the adhesive bead is contiguous with the adhesive.

29. A method for forming a disposable garment comprising:
simultaneously, applying a securing element to an inner layer as the inner layer is being unwound, unwinding an outer layer and oscillating elastic filaments between the inner layer and outer layer;
placing the inner layer on top of the outer layer, the outer layer having elastic filaments contained therein, to produce a laminate; and
cutting the elastic filaments located outside the laminate with a cutting means wherein the elastic filaments snap back to an outer edge of the securing element.

30. The method of claim 29 wherein the securing element is an adhesive bead.

31. The method of claim 30 wherein the adhesive bead is a continuous bead about 0.2 to 0.6 cm wide.

32. The method of claim 30 wherein the adhesive bead is applied at rate of about five (5) to 100 gsm.

33. The method of claim 29 wherein the elastic filaments are oscillated in a substantially sinusoidal pattern.

34. The method of claim 29 wherein the cutting means is selected from the group consisting of a pinch flex cutter, ultrasonics and heat laser.

35. The method of claim 29 wherein the disposable garment is an absorbent article.
